(19) United States
(12) Patent Application Publication
(10) Pub. No.: US 2024/0033426 A1
(43) Pub. Date: Feb. 1, 2024

(54) INFUSION PUMP WITH OCCLUSION DETECTION

(71) Applicant: Smiths Medical ASD, Inc., Plymouth, MN (US)

(72) Inventors: Sameer PAI, Plymouth, MN (US); Paul Harrison COONS, Edina, MN (US); Kevin KRAUTBAUER, Inver Grove Heights, MN (US); Henry MADDEN, Minneapolis, MN (US); Erika SMITH, Plymouth, MN (US); Sarah THOLE, Minneapolis, MN (US); Anushrut JIGNASU, Ames, IA (US)

(21) Appl. No.: 18/256,109

(22) PCT Filed: Nov. 15, 2021
(86) PCT No.: PCT/US2021/059369

§ 371 Date: Jun. 6, 2023

Related U.S. Application Data
(60) Provisional application No. 63/199,140, filed on Dec. 9, 2020.

(51) Int. Cl. A61M 5/168; A61M 5/172; A61M 5/145; G16H 20/17; G16H 40/40

(52) U.S. Cl. CPC A61M 5/16854; A61M 5/1723; A61M 5/1452; G16H 20/17; G16H 40/40; A61M 2205/332

(57) ABSTRACT

A system for occlusion detection, including a cartridge containing an infusate, a plunger driven by a drive mechanism configured to be advanced within the cartridge to expel infusate from the cartridge, a force sensor configured to sense an actual force exerted by the plunger, and a control unit configured to determine an estimated force based on an expected decay in frictional force between the plunger and the cartridge for comparison to the actual force sensed by the force sensor, wherein a deviation between the estimated force and the actual force exceeding a threshold triggers an occlusion alarm.

### TECHNICAL FIELD

The present disclosure relates generally to infusion pump systems, and more particularly to systems and methods for occlusion detection.

### BACKGROUND

Various types of infusion pumps have been useful for managing the delivery and dispensation of a prescribed amount or dose of a drug, fluid, fluid like substance, or infusate (herein, collectively, an “infusate”) to patients. Infusion pumps provide significant advantages over manual administration by accurately delivering infusates over an extended period of time. Infusion pumps are particularly useful for treating diseases and disorders that require regular pharmacological intervention, including cancer, diabetes, and vascular, neurological, and metabolic disorders. Infusion pumps also enhance the ability of healthcare providers to deliver anesthesia and manage pain. Infusion pumps are used in various settings, including hospitals, nursing homes, and other short-term and long-term medical facilities, as well as in residential care settings. There are many types of infusion pumps, including ambulatory, large-volume, patient-controlled anesthesia (PCA), elastomeric, syringe, enteral, and insulin pumps. Infusion pumps can be used to administer medication through a variety of delivery methods, including intravenously, intraperitoneally, inter-arterially, intradermally, subcutaneously, in close proximity to nerves, and into an inter-operative site, epidural space, or subarachnoid space.

In the field of medicament delivery devices and systems including so-called “syringe pumps,” typically a prefilled medication syringe is mechanically driven under microprocessor control to deliver a prescribed dose of medicament at a controlled rate to a patient through an infusion line fluidly connected to the syringe. Syringe pumps typically include a motor that rotates a lead screw. The lead screw in turn activates a plunger driver, which forwardly pushes a thumb-press of a plunger within the barrel of the syringe. Pushing the plunger thus forces the dose of medicament outwardly from the syringe, into the infusion line, and into the patient intravenously. Examples of syringe pumps are disclosed in, for example, U.S. Pat. No. 8,182,461 titled “Syringe Pump Rapid Occlusion Detection System” and U.S. Pat. No. 10,004,847B2 titled “Occlusion Detection,” the contents of which are incorporated by reference herein. As used throughout this disclosure, the term “syringe pump” is intended to generally pertain to any device which acts on a syringe to controllably force fluid outwardly therefrom.

As a subset of syringe pumps, one type of pump that has been developed is a micro-infusion pump (alternatively referred to as a “burst pump”). Micro-infusion pumps are small, typically ambulatory pumps, which may be carried under a patient's clothing or otherwise very near to an injection site on the patient. Micro-infusion pumps are capable of reliably delivering low infusate flow rates and are often used for multiple consecutive days. In some cases, the pumps utilize replaceable syringe-like cartridges, into which a plunger advances to administer the infusate. In order to maintain a long battery life, micro-infusion pump systems typically deliver infusate in relatively short or small increments or “bursts” representing very small advances of the plunger within the cartridge (e.g., 3 μm or less), with relatively long, low power consumption pauses between the bursts.

While various types of syringe pumps have been used in medical environments for years, manufacturers of these devices continually strive for more efficient, effective and safer usage. In some cases, infusion pumps such as traditional syringe pumps and micro-infusion pumps can experience an occlusion. In the medical arts, the term “occlusion” typically refers to the blocking or restriction of a normally open passage. In some instances, an occlusion is desired such as when a catheter is pinched off or temporarily collapsed into a closed state intentionally by a practitioner during a medical procedure. In other instances, an unintended occlusion might occur when the intended and commanded forward progression of the plunger through the syringe or cartridge barrel is blocked or an intended outward flow of infusate from a pump to a patient is otherwise impeded, as when for example the infusion line tubing is kinked or otherwise structurally blocked to some degree. If the occlusion is not noticed, the patient likely would not receive the prescribed infusate leading to potentially serious consequences.

Attempts to sense or detect occlusions in medical devices have therefore been made. For example, some syringe pumps detect occlusions by use of a pressure sensor that senses a force exerted by the aforementioned syringe thumb-press on the plunger driver. When the force experienced by the pressure sensor exceeds a predetermined threshold force, a processor connected to the pressure sensor generates a signal indicating that an occlusion has possibly occurred or is possibly occurring. Nevertheless, further advances in occlusion detection are desired. The present disclosure addresses one or more of these concerns.

### SUMMARY OF THE DISCLOSURE

Occlusion detection systems and methods described in detail, by way of examples herein, make novel and inventive use of input parameters, such as variations in a frictional force and other factors, between a plunger and syringe cartridge during operational use. In some embodiments, such compensation for expected changes in the frictional force advantageously results in more accurate pressure detection, a reduction in the time necessary to identify the presence of an occlusion, and a higher degree of confidence in occlusion sensing.

An embodiment of the present disclosure presents a system for occlusion detection, including a cartridge containing an infusate, a drive mechanism configured to advance a plunger within the cartridge to expel infusate from the cartridge, a force sensor configured to sense an actual force exerted by the plunger, and a control unit configured to determine an estimated force based on an expected decay in frictional force between the plunger and the cartridge for comparison to the actual force sensed by the force sensor, wherein a deviation between the estimated force and the actual force exceeding a threshold triggers an occlusion alarm.

In an embodiment, the estimated force is based on a mathematical model used to predict an expected decay in frictional force between drive mechanism activations. In an embodiment, the mathematical model is a two-exponential equation. In an embodiment, the mathematical model is represented by the equation F(t)=C₀+C₁\hat{ }(−((t−t₀))/t₁)+C₂\hat{ }(−((t−t₀))/t₂). In an embodiment, the estimated force is based on one or more input parameters measurable by the system. In an embodiment, the input parameters include at least one of the actual force sensed by the force sensor at the end of a drive mechanism activation and/or one or more material compliances of the system.

Another embodiment of the present disclosure presents a system for occlusion detection, including a cartridge containing an infusate, a drive mechanism configured to advance a plunger within the cartridge to expel infusate from the cartridge, a force sensor configured to sense an actual force exerted by the plunger, and a control unit configured to plot a trend line by connecting a force sensed by the force sensor at some instant in time within a burst cycle across a sequence of burst cycles, wherein a trend line slope above a threshold triggers an occlusion alarm. In an embodiment, each burst cycle is represented by a period of time between drive mechanism activations, beginning and ending at a cessation of the drive mechanism activations. In an embodiment, the trend line is fit to an average of the sensed pressure at a cessation of drive mechanism activations. In an embodiment, a positive slope of the trend line is indicative of an occlusion.

Another embodiment of the present disclosure provides a method of occlusion detection, comprising: estimating a system compliance; computing a probability of a system occlusion; and triggering at least one of an occlusion alert, alarm or notification upon determining that the computed probability of a system occlusion exceeds a threshold. In an embodiment, the system compliance is computed by measurement of a pressure reduction after cessation of a drive mechanism activation. In an embodiment, the system compliance is computed as a function of an expected decay in frictional force. In an embodiment, the system compliance is computed by dividing a change in pressure as measured by a force sensor, by a change in infusate volume. In an embodiment, computing a probability of a system occlusion involves Bayesian Inference.

The summary above is not intended to describe each illustrated embodiment or every implementation of the present disclosure. The figures and the detailed description that follow more particularly exemplify these embodiments.

### BRIEF DESCRIPTION OF THE DRAWINGS

The disclosure can be more completely understood in consideration of the following detailed description of various embodiments of the disclosure, in connection with the accompanying drawings, in which:

FIG. 1 is a perspective view depicting an infusion pump system attached to a patient, in accordance with an embodiment of the disclosure.

FIG. 2 is an exploded, perspective view depicting an infusion pump, in accordance with an embodiment of the disclosure.

FIG. 3 is a graphical plot depicting an increase in a frictional force during advancement of a plunger within a cartridge, in accordance with an embodiment of the disclosure.

FIG. 4 is a graphical plot depicting a natural decay in the frictional force between plunger “bursts,” in accordance with an embodiment of the disclosure.

FIG. 5 is a graphical plot depicting a trend line across sensed force readings at some instant in time within each burst cycle across a sequence of burst cycles, in accordance with an embodiment of the disclosure.

FIG. 6 is a flowchart depicting a method of occlusion detection using Bayesian Inference, in accordance with an embodiment of the disclosure.

FIG. 7A is a graphical plot depicting distribution curves of cartridge compliance and system compliance, in accordance with an embodiment of the disclosure.

FIG. 7B is a graphical plot depicting an average distribution curve combining the cartridge compliance and system compliance of FIG. 7A, in accordance with an embodiment of the disclosure.

While embodiments of the disclosure are amenable to various modifications and alternative forms, specifics thereof shown by way of example in the drawings will be described in detail. It should be understood, however, that the intention is not to limit the disclosure to the particular embodiments described. On the contrary, the intention is to cover all modifications, equivalents, and alternatives falling within the spirit and scope of the subject matter as defined by the claims.

### DETAILED DESCRIPTION

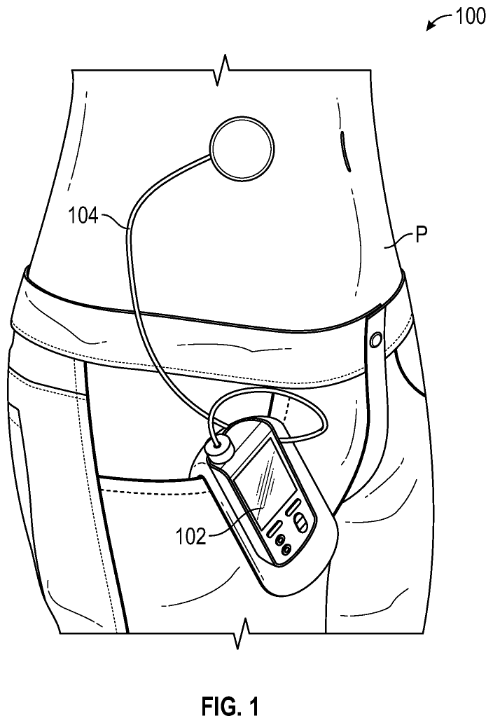

Referring to FIG. 1, an infusion pump system 100 for administering infusate to a patient (P) is depicted in accordance with an embodiment of the disclosure. The infusion pump system 100 can include an infusion pump 102 configured to control delivery of infusate to the patient P via an infusion set 104 or other tubing fluidly coupled between the pump 102 and the patient P.

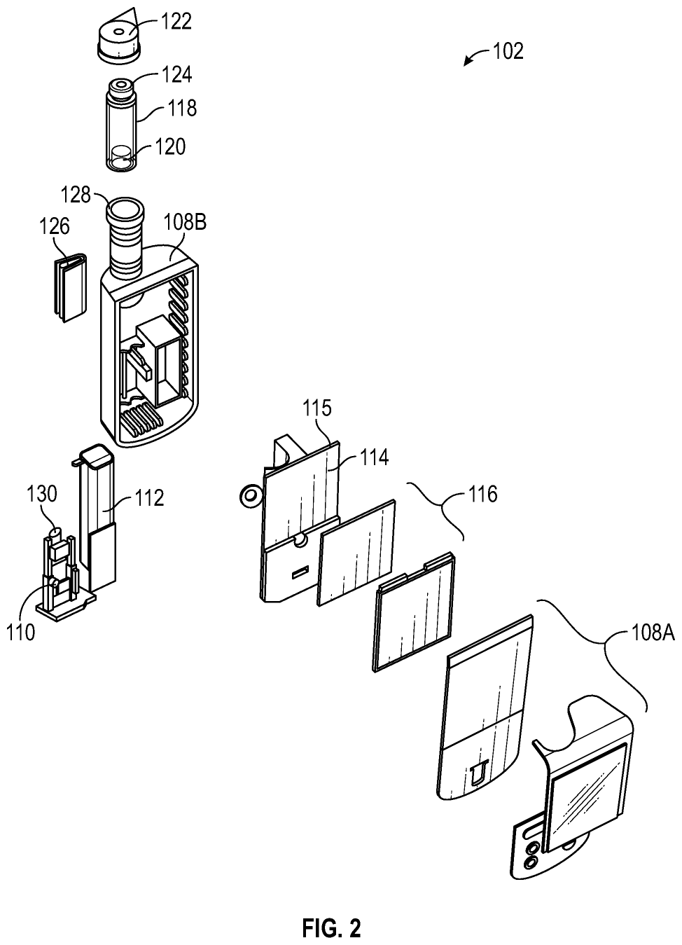

Referring to FIG. 2, an exploded, perspective view of the infusion pump 102 is depicted in accordance with an embodiment of the disclosure. In an embodiment, the infusion pump 102 can include a front housing 108A and rear housing 108B configured to provide a chassis to which other components of the infusion pump 102, such as a drive mechanism 110, power source 112, control unit 114, memory 115, and graphical user interface 116, can be assembled.

The infusion pump 102 can further include or be operably coupled to a cartridge 118 containing an infusate, which can include a plunger 120 for expulsing the infusate therefrom. The cartridge 118 can be any suitable syringe-like object, container, vessel or other source containing or supplying a quantity of infusate. In some embodiments, the cartridge 118 can be selectively removed and replaced as needed, for example upon depletion of a supply of infusate therein. An infusion set connector 122 can fluidly couple a dispensing end 124 of the cartridge 118 to the infusion set 104.

The plunger 120 can be driven by the drive mechanism 110, for example via a lead screw arrangement configured to cooperatively actuate the plunger 120, thereby driving fluid from the cartridge 118. The drive mechanism 110 can be powered by the power source 112. In some embodiments, the power source 112 can be in the form of one or more disposable or rechargeable batteries, which can be selectively removed and replaced via a battery door 126. The drive mechanism 110 can be controlled via the control unit 114.

The control unit 114 can be any suitable programmable device that accepts digital data as input, is configured to process the input according to instructions or algorithms, and provides results as outputs. In an embodiment, the control unit 114 can be a central processing unit (CPU) configured to carry out the instructions of a computer program. In an embodiment, the control unit 114 can be an advanced RISC (Reduced Instruction Set Computing) Machine (ARM) processor or other embedded microprocessor. In an embodiment, the control unit 114 comprises a multi-processor cluster. Control unit 114 can therefore be configured to perform at least basic arithmetical, logical, and input/output operations.

The memory 115 can comprise volatile or nonvolatile memory as required by the coupled control unit 114 to not only provide space to execute the instructions or algorithms, but to provide the space to store the instructions themselves. In embodiments, volatile memory can include random-access memory (RAM) dynamic random access memory (DRAM) or static random access memory (SRAM) for example. In embodiments, nonvolatile memory can include read-only memory, flash memory, ferroelectric RAM, hard disk, floppy disk, magnetic tape, or optical disk storage, for example. The foregoing examples in no way limit the type of memory that can be used, as the embodiments are given only by way of example and are not intended to limit the subject matter hereof.

The control unit 114 can receive inputs from the graphical user interface 116, which in an embodiment can be a touchscreen input and display system. In an embodiment, the control unit 114 can be in communication with an antenna 128 configured to send and receive data wirelessly to one or more external computing devices, such as a mobile computing platform (e.g., smart phone, tablet, personal computer, etc.) and/or a network. In an embodiment, the antenna 128 can be an RFID coil; although other types of antennas are also contemplated.

In an embodiment, the control unit 114 can additionally receive inputs from other input devices, sensors and monitors, such as a sensor 130. In some embodiments, the sensor 130, which can be positioned in-line with the drive mechanism 110, can be configured to monitor a force between the drive mechanism 110 and the plunger 120, and/or the position of the plunger 120 relative to the cartridge 118 according to system specifications. The sensor 130 can comprise a force sensor, pressure sensor, distance sensor, proximity sensor, or any other suitable sensor. In some embodiments, the sensor 130 can additionally function as an occlusion detection sensor configured to sense when a fluid pressure of the infusate exceeds a predefined threshold, thereby indicating the likelihood of an occlusion in the cartridge 118 and/or infusion set 104.

The infusion pump 102 depicted in FIGS. 1 and 2 is an example of an ambulatory type of pump that can be used to deliver a wide range of therapies and treatments. Such ambulatory pumps can be comfortably worn by or otherwise removably coupled to a user for in-home ambulatory care by way of belts, straps, clips or other simple fastening mechanisms; and can also be alternatively provided on an ambulatory pole mounted arrangement within hospitals and other medical care facilities.

In an embodiment, the infusion pump 102 can be a burst or micro-infusion pump configured to provide intermittent infusions of small doses of medication over an extended period of time. In a non-limiting embodiment, the infusion pump system 100 can be configured to administer treprostinil (marketed under the name Remodulin® by United Therapeutics Corporation), or other medications for the treatment of pulmonary arterial hypertension (PAH), either subcutaneously or intravenously; although the administration of infusates other than treprostinil is also contemplated.

The embodiment of the infusion pump 102 depicted in FIGS. 1 and 2 is provided only by way of example, and is not intended to limit the scope of the subject matter herein. Other types of pumps and other pump configurations can be utilized in various embodiments. Additionally, it should be appreciated that the systems and methods as described herein, particularly those configured to identify an occlusion between a plunger and an infusate vessel or cartridge, can equally be applied to other types of infusion pumps, particularly syringe pumps and other types of infusion pumps configured to administer infusate via an advancing plunger within a syringe, cartridge or other vessel containing infusate, such as those disclosed in, for example, U.S. Pat. No. 8,182,461 titled “Syringe Pump Rapid Occlusion Detection System” and U.S. Pat. No. 10,004,847B2 titled “Occlusion Detection,” which were previously incorporated by reference.

Occlusion detection is typically performed by estimating the pressure within the cartridge 118 and/or infusion set 104 and issuing an alert, alarm or notification when the value exceeds a set limit. With low medicament flow rates, such as that in micro-infusion or burst pumps, such conventional methods can lead to excessively long times until occlusion detection. Even after an occlusion has been detected, and the drive mechanism 110 has been stopped, the pressure within the infusion set 104 between the occlusion and the drive mechanism 110 will remain at a heightened pressure (e.g., the pressure at the time that the drive mechanism 110 stops). A sudden release of the occlusion (e.g., the infusion line suddenly becoming unkinked) could cause the pressurized fluid to be delivered to the patient in a large bolus of infusate, which with some types of infusates can be dangerous. Embodiments of the present disclosure address these concerns.

### 1. Parameter Dependent Friction Models

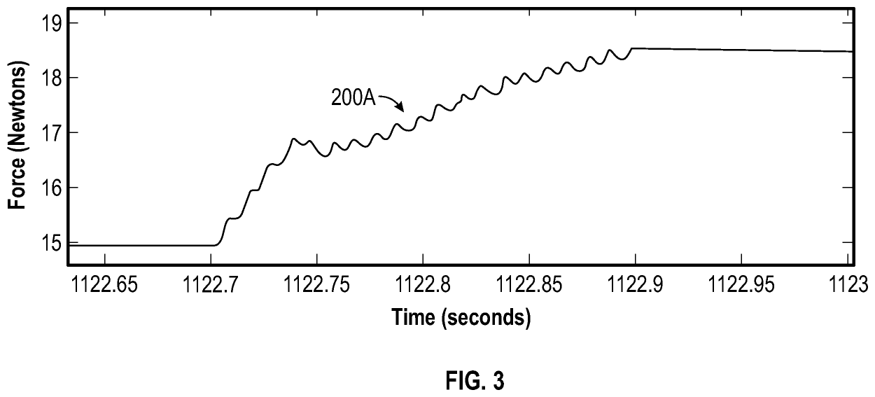

One significant source of error in pressure estimation for burst pumps is the variation of friction, typically experienced between a wall of the cartridge 118 and the plunger 120. Variations in frictional force while the plunger 120 is being advanced very according to a number of factors including, but not limited to plunger 120 travel distance during the current “burst”, material compliance, and the normal force exerted on the plunger 120. Referring to FIG. 3, a graph showing an example of an increase in the frictional force 200A during advancement of the plunger 120 within the cartridge 118 is depicted in accordance with an embodiment of the disclosure. As depicted in the graph of FIG. 3, the x-axis shows the time (in seconds), while the y-axis shows the force (in Newtons).

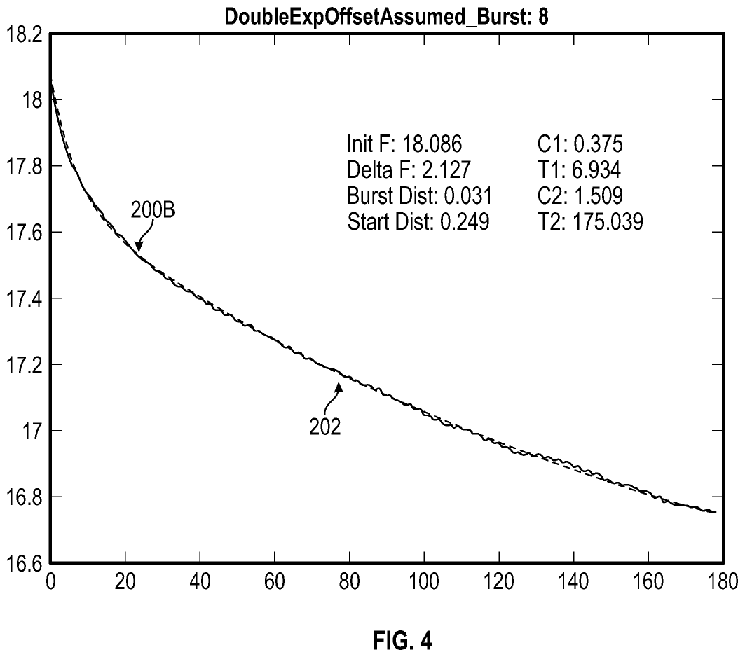

When the plunger 120 stops moving (e.g., at the completion of the “burst”) the frictional force buildup during advancement of the plunger 120 naturally decays over time. Both the rate and magnitude of decay very based on a number of factors, including, but not limited to, material properties, distance traveled, time, and other factors. Referring to FIG. 4, a graph showing an example of decay in the frictional force 200B between plunger “bursts” is depicted in accordance with an embodiment of the disclosure. As depicted in the graph of FIG. 4, the x-axis shows the time (in seconds), while the y-axis shows the force (in Newtons).

In some embodiments, a curve 202 can be fit to an actual, measured decay in frictional force 200B, wherein the curve 202 can be represented by a mathematical model. Thereafter, the mathematical model can be used to predict a future or expected decay in frictional force between plunger bursts. For example, in one embodiment, the mathematical model can be represented by the following two-exponential equation:

$$F ( t ) = {C}_{0} + {C}_{1} {e}^{- \frac{( t - {t}_{0} )}{{t}_{1}}} + {C}_{2} {e}^{- \frac{( t - {t}_{0} )}{{t}_{2}}}$$

Wherein F(t) represents the frictional force value (e.g., corresponding to curve 202) at some instant in time t, occurring after the motor has stopped t₀, and wherein C₀, C₁, and C₂ represent constants which can be determined through a variety of mathematical processes to curve fit the expected decay in frictional force overtime t to an actual, measured or experimental decay in frictional force.

Although, the expected frictional force F(t) is expressed as a function of time t, there are a large number of input parameters accounted for in determining constants C₀, C₁, and C₂. For example, as previously mentioned, these input parameters can include the distance the plunger 120 travels during the previous burst, the normal force exerted on the plunger 120 at the end of the burst, and the material compliance of the system (e.g., material properties of the plunger 120, cartridge 118, and/or extension set 104), among other factors.

In some embodiments, each of the constants C₀, C₁, and C₂ can be written in terms of a function of these input parameters. For example, C₀ can be represented by:

C₀(d,F_{burst}, . . . )

Wherein, d represents the distance the plunger 120 travels during the previous burst, F_{burst} represents the normal force exerted on the plunger 120 at the end of the burst, and ( . . . ) represents other input parameters taken into account in determining constant C₀. In cases where certain input parameters (e.g., the distance the plunger 120 travels (d) and the normal force exerted on the plunger 120 at the end of the burst (F_{burst})) are measurable by the system, the expected frictional force F(t) at any given instant in time t can be expressed in terms of a function of both time t and the measured input parameters. For example, in some embodiments, the expected frictional force F(t) can be represented by:

$$F ( t ) = {C}_{0} ( d , {F}_{burst , …} ) + {C}_{1} ( d , {F}_{burst , …} ) {e}^{- \frac{( t - {t}_{0} )}{{t}_{1} ( d , {F}_{burst , …} )}} + {C}_{2} ( d , {F}_{burst , …} ) {e}^{- \frac{( t - {t}_{0} )}{{t}_{2} ( d , {F}_{burst , …} )}}$$

Accordingly, in some embodiments, determination of the expected frictional force decay F(t) (e.g., corresponding to curve 202) can use known or estimated relationships between input parameters known to influence frictional force decay between burst cycles. Inclusion of these input parameters in the prediction of the frictional force decay can lead to more accurate pressure detection, which can in turn lead to clinical benefits of reduced nuisance alarms and reduced time to occlusion.

2. Occlusion Detection by Measurement of Pressure Reduction after Motor Halt

In some embodiments, the expected frictional force decay 202 can be used as an aid in determining a natural fluctuation in cartridge 118 pressure during operation. For example, in some embodiments, the infusion pump 102 can include a force sensor 130 configured to sense a force experienced by the plunger 120. According to the formula P=F/A, dividing the sensed force by the area within the cartridge cylinder 118 provides a pressure within the cartridge cylinder 118. Factoring in an actual or expected decay in frictional force (the equal and opposite reaction of which should be measurable by the force sensor 130) can provide a clearer understanding of an expected change in pressure within the cartridge cylinder 118 over time.

When the motor 110 of an infusion pump 102 stops moving (e.g., at the completion of a burst), any pressure buildup within the cartridge 118 generally results in a flow of fluid out of the cartridge 118 and an associated extension set 104, and into a patient, thereby reducing the pressure within the cartridge 118. If enough time passes between bursts, absent an occlusion, the pressure within the cartridge 118 will reduce to an equilibrium pressure (e.g., determined by the fluid pressure or tissue resistance of the patient) upon which further flow of fluid out of the cartridge 118 ceases. However, if the system 100 is occluded the pressure will only reduce slightly (e.g., as a result of material compliance and a decay in frictional force), never reaching the ambient, equilibrium pressure.

As briefly described above, the system can be configured to monitor a pressure within the cartridge 118 (e.g., via a sensor 130) over a period of time following cessation of the motor 110 (e.g., completion of a burst), with the goal of determining whether any reduction in pressure is characteristic of an occluded or un-occluded system 100. In some embodiments, determining whether any reduction in pressure is characteristic of an occluded or un-occluded system can be done by comparing an actual change in pressure to an expected change in pressure, which can be determined based on an expected decay in frictional force.

In some embodiments, determining whether any reduction in pressure is characteristic of an occluded or un-occluded system 100 can be done either by comparing the relaxation amount against some expected relaxation amount (e.g. that predicted by the expected decay in frictional force) or by using the information to compute a characteristic system parameter, in this case the system compliance. System compliance can be computed according to the following formula:

$${β}_{est} = \frac{Δ {F}_{relax} - Δ {F}_{measured}}{Δ V {A}_{cartridge}}$$

Wherein, βₑₛₜ is the estimated system compliance, ΔFᵣₑₗₐₓ is the change in frictional force (e.g., as previously described), ΔF_{measured} is the change in the measured or sensed plunger force, ΔV is the volume delivered in the burst, and A_{cartridge} is the area of the cartridge 118. The estimated system compliance can then be compared against expected values, either directly or through Bayesian Inference. In some embodiments, determining whether any reduction in pressure is characteristic of an occluded or un-occluded system 100 according to this method can result in a reduction in time until determination of an occlusion is made, as well as a reduction in the size of any bolus administered to a patient if the occlusion would rather suddenly be cleared.

### 3. Occlusion Detection by Trending Pressure

Occlusion detection by conventional methods (e.g., sensing a pressure within the cartridge 118 and/or administration set 104 and providing an occlusion alarm when the sensed pressure exceeds a predefined limit) can lead to excessively long times until the determination of an occlusion is made, particularly with low flow rates. Additionally, by the time that a determination of an occlusion has been made, a quantity of pressurized infusate trapped in the cartridge 118 and/or administration set 104 over time needed to determine that an occlusion is present may result in the delivery of a large bolus to the patient upon removal of the occlusion.

In an effort to reduce the period of time required to determine the presence of an occlusion, in some embodiments, the system 100 can use an optimized slope-based method for determining the presence of an occlusion, wherein as pressure within the cartridge 118 and/or administration set 104 fluctuates during operation, a trending pressure increase (e.g., as measured at one point in an operation cycle of system 100) can trigger an occlusion alarm.

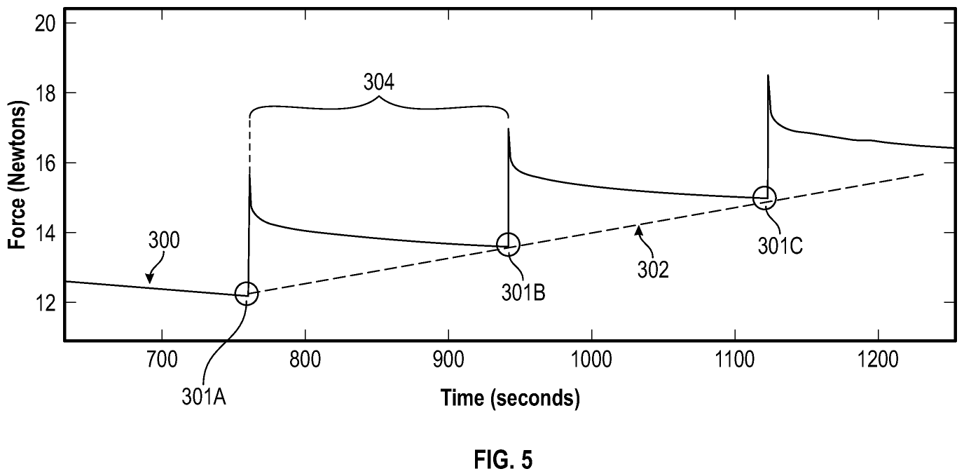

For example, referring to FIG. 5, a trend-line 302 can be created during pump operation by connecting a sensed force reading 300 at some instant in time within each burst cycle 304 across a sequence of burst cycles. In some embodiments, sensed force readings 301A-C can be taken just prior to the bursts to reduce noise in the sensed force. Noise in the sensed force is most likely to occur between power cycles (e.g., during the normal decay in fluid pressure within the cartridge 118 and/or administration set 104). A trend line 302 with a positive slope above a given threshold may be indicative of an occlusion.

In some embodiments, the change in pressure can be divided by the volume of infusate delivered between measurements, according to the following formula:

$${β}_{est} = \frac{Δ P}{Δ V}$$

Dividing the change in pressure by the change in volume can have the benefit of normalizing the pressure change. In some embodiments, the normalized pressure change can also be the equivalent of the estimated system compliance used to predict occlusions.

In some embodiments, monitoring a trending pressure 302 to determine if the pressure within the cartridge 118 and/or administration set 104 at a given instant in time within the burst cycle 304 is generally increasing during operation can result in a reduction in time until a determination of an occlusion is made, as well as a reduction in the size of any bolus administered to a patient if the occlusion would rather suddenly be cleared.

### 4. Occlusion Detection by Bayesian Inference

In some embodiments, a probability or likelihood that an occlusion is occurring (or that an occlusion alarm is about to be triggered) can be computed according to a Bayesian Inference algorithm. Bayesian Inference is a formal expression of “educated guessing” where the likelihood of a condition or event is estimated given some data that is received into an algorithm. In some embodiments, a Bayesian Interference algorithm can be used to determine whether the system is occluded based on data received about one or more known system parameters (e.g., system compliance). In some embodiments, a Bayesian Inference algorithm can reduce nuisance alarms and the time needed to trigger an occlusion alarm by providing a logical method of bringing additional knowledge about the system into the occlusion detection method.

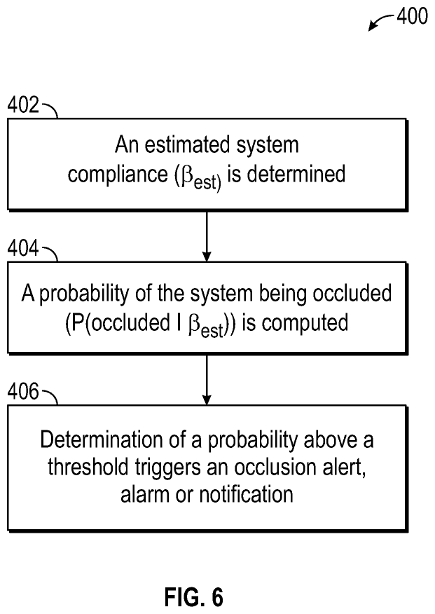

Referring to FIG. 6, a flowchart depicting a method of occlusion detection 400 using Bayesian Inference, is depicted in accordance with an embodiment of the disclosure. At 402, an estimated system compliance (est) is determined. For example, in an embodiment, the estimated system compliance can be calculated according to at least one of a measurement of pressure reduction after motor halt, a trending pressure, and/or by some other method.

With the estimated system compliance (βₑₛₜ), at 404, a probability of the system being occluded (P(occluded| est)) is computed. During this step, additional information about the system (e.g., material compliance of the system components) can be used to improve the detection of an actual occlusion. For example, if the occlusion occurs in close proximity to an outlet of a cartridge, the estimated system compliance will be approximately equal to the compliance of the cartridge alone (e.g., the “cartridge compliance”). If, however, the occlusion occurs in proximity to an end the infusion set, the estimated system compliance will be approximately equal to the compliance of both the cartridge and the infusion set (e.g., the “system compliance”).

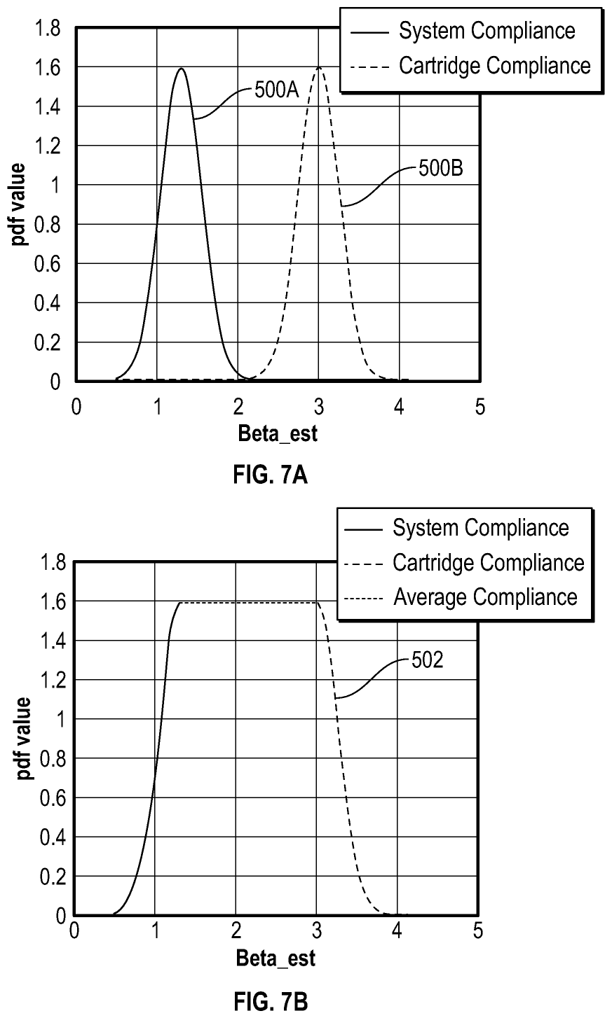

With additional reference to FIG. 7A, factoring in some standard deviation of error (e.g., to account for material changes over ranges of temperatures, etc.), distribution curves of cartridge compliance 500A and system compliance 500B are plotted adjacent to one another. As depicted in the graph of FIG. 7A, the y-axis shows a range of compliance values, while the x-axis shows the probability of measuring any particular compliance value in the event of an occlusion in proximity to the outlet of, for example, the cartridge 118 and in proximity to an end of, for example, the infusion set 104, respectively. Given that there is usually no way to know with certainty where in a system an occlusion may possibly occur, the actual compliance value of an occlusion could take any value between the two distributions 500A-B. Accordingly, with additional reference to FIG. 7B, the probability or likelihood of the compliance value of any given occlusion occurring between the cartridge compliance 500A and the system compliance 500B can be assumed to be the average of the two peaks.

Accordingly, in an embodiment, the probability or likelihood that the system 100 is occluded can be calculated according to the following formula:

$$P ( occluded \vert {β}_{est} ) = \frac{P ( {β}_{est} \vert occluded ) * P ( occluded )}{P ( {β}_{est} )}$$

Wherein “|” reads “given,” such that the left-hand side of the equation (i.e., P (occluded|βₑₛₜ)) represents the probability that the system is actually occluded given the estimated system compliance (βₑₛₜ) determined at step 402. Further, wherein P(occluded) is a previously computed P (occluded|βₑₛₜ) or some initial starting value. That is, initially, a starting value can be assumed for P(occluded), thereafter the previously calculated P (occluded|βₑₛₜ) can be used when a new βₑₛₜ measurement is made. P(βₑₛₜ|occluded) is the probability determined in the previous step, and P(βₑₛₜ) is a normalizing factor that is used to control reactivity of the algorithm.

Accordingly, in some embodiments, the probability that the system 100 is occluded P (occluded|βₑₛₜ) represents the likelihood of determining the system compliance value βₑₛₜ (at step 502), if the system 100 was known to have an occlusion somewhere between the outlet of the cartridge 118 and the end of the infusion set 104. At 406, determination of a probability above a threshold (e.g., equal or greater to about 0.85) can trigger an occlusion alert, alarm or notification. Determination of a probability below the threshold can be regarded as noise.

Notable advantages of using a Bayesian Inference method is that the system information (e.g., characterized distribution of compliances, system pressures or other measurable characteristics), can be used to reduce the noise in the system by disregarding systems measurements that are not reasonable either because they are too low to be characteristic of an occlusion or too high so that they are characteristic of spurious noise. Accordingly, the Bayesian Inference method can be used to reduce nuisance alarms and/or to reduce the time need to make a determination of an occlusion (as well as a reduction in the size of any bolus administered to a patient if the occlusion would rather suddenly be cleared). Advantageously, the Bayesian Inference method also enables multiple occlusion algorithms to function together, as any algorithm that estimates the βₑₛₜ value can be used to update the current probability estimate.

While systems for, and methods of, occlusion detection have been particularly shown and described with reference to the accompanying figures and specification, it should be understood however that other modifications thereto are of course possible; and all of them are intended to be within the true spirit and scope of the novel and inventive systems and methods described herein. Thus, configurations and designs of various features could be modified or altered depending upon particular embodiments.

Various embodiments of systems, devices, and methods have been described herein. These embodiments are given only by way of example and are not intended to limit the scope of the claimed subject matter. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed subject matter.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. A system for occlusion detection, comprising:
a cartridge containing an infusate;
a plunger driven by a drive mechanism configured to be advanced within the cartridge to expel infusate from the cartridge;
a force sensor configured to sense an actual force exerted by the plunger; and
a control unit configured to determine an estimated force based on an expected decay in frictional force between the plunger and the cartridge for comparison to the actual force sensed by the force sensor, wherein a deviation between the estimated force and the actual force exceeding a threshold triggers an occlusion alarm.

2. The system of claim 1, wherein the estimated force is based on a mathematical model used to predict an expected decay in frictional force between drive mechanism activations.

3. The system of claim 2, wherein the mathematical model is a two exponential equation.

4. The system of claim 1, wherein the mathematical model is represented by the equation
F(t)=C₀+C₁\hat{ }(−((t−t₀))/t₁)+C₂\hat{ }(−((t−t₀))/t₂).

5. The system of claim 1, wherein the estimated force is based on one or more input parameters measurable by the system.

6. The system of claim 1, wherein the input parameters include at least one of the actual force sensed by the force sensor at the end of a drive mechanism activation and/or one or more material compliances of the system.

7. A system for occlusion detection, comprising:
a cartridge containing an infusate;
a plunger driven by a drive mechanism configured to be advanced within the cartridge to expel infusate from the cartridge;
a force sensor configured to sense an actual force exerted by the plunger; and
a control unit configured to plot a trend line by connecting a force sensed by the force sensor at some instant in time within a burst cycle across a sequence of burst cycles, wherein a trend line slope above a threshold triggers an occlusion alarm.

8. The system of claim 7, each burst cycle is represented by a period of time between drive mechanism activations, beginning and ending at a cessation of the drive mechanism activations.

9. The system of claim 7, wherein the trend line is fit to an average of the sensed pressure at a cessation of drive mechanism activations.

10. The system of claim 7, wherein a positive slope of the trend line is indicative of an occlusion.

11. A method of occlusion detection, comprising:
estimating a system compliance;
computing a probability of a system occlusion; and
triggering at least one of an occlusion alert, alarm or notification upon determining that the computed probability of a system occlusion exceeds a threshold.

12. The method of claim 11, wherein the system compliance is computed by measurement of a pressure reduction after cessation of a drive mechanism activation.

13. The method of claim 11, wherein the system compliance is computed as a function an expected decay in frictional force.

14. The method of claim 11, wherein the system compliance is computed by dividing a change in pressure as measure by a force sensor, by a change in infusate volume.

15. The method of claim 11, wherein computing a probability of a system occlusion involve Baysian interference.
